(19) United States
(12) Patent Application Publication
(10) Pub. No.: US 2022/0368855 A1
(43) Pub. Date: Nov. 17, 2022

(54) VIRTUAL WAITING ROOM

(71) Applicant: MEND VIP, Inc., Orlando, FL (US)

(72) Inventors: Matthew D. MCBRIDE, Orlando, FL (US); Brandon LASSITER, Orlando, FL (US); Jaime Andres CLAVIJO MENDIETA, Medellin (CO); Jack VAN STONE, Orlando, FL (US); Jessica NEYER, Sherman Oaks, CA (US); Rogelio MENDIOLA, Orlando, FL (US); Tejal SHAH, Winter Garden, FL (US)

(73) Assignee: MEND VIP, Inc., Orlando, FL (US)

(21) Appl. No.: 17/317,463

(22) Filed: May 11, 2021

(51) Int. Cl. H04N 7/15; H04L 29/06; H04L 12/58; G16H 40/20; G06Q 30/00; G06Q 30/02

(52) U.S. Cl. CPC H04N 7/157; H04L 65/1089; H04L 65/1093; H04L 51/02; G16H 40/20; G06Q 30/016; G06Q 30/0277; G16H 10/60

(57) ABSTRACT

An enhanced virtual waiting room may be provided. The enhanced virtual waiting room may be presented to a user while waiting for a virtual appointment to start. The enhanced virtual waiting room may test and troubleshoot a users' connection. The enhanced virtual waiting room may also provide curated content and advertisements to a user.

### BACKGROUND

The distribution of health-related services and information through electronic telecommunication technologies has increased significantly with recent developments in audio and video teleconferencing technology. For example, doctors are able to perform virtual visits with patients using audio and video conferencing technology. The virtual visits may take place with one or more of a service provider and patient/customer being remotely located or with a service provider and patient/customer being located in different rooms within an office space. However, like in-person appointments, patients/customers may sign on to an appointment prior to the appointment time and service providers schedules may not allow them to join a scheduled appointment at its scheduling starting time. Therefore, patients or customers are often left on hold or staring at a blank screen awaiting connection to their provider resulting in a previously untapped captive audience.

Recent data from leading telehealth providers suggests over 400,000 video visits per month with an average wait time of over 20 minutes per visit. It therefore may be desirable to provide information, tasks, advertisements or entertainment for customers and patients awaiting a provider appointment over teleconferencing technology, such as a video or web-conferencing platform.

Furthermore, information communicated to or from a customer/patient may be of a sensitive nature, so sufficient security precautions must be taken to safeguard the information and comply with government regulations. For example, such systems must comply with the requirements of the Health Insurance Portability and Accountability Act of 1996 (“HIPAA”), as well as the Health Information Technology for Economic and Clinical Health Act, enacted under Title XIII of the American Recovery and Reinvestment Act of 2009 (“HITECH”).

### SUMMARY

According to an exemplary embodiment, a method for engaging users waiting for a virtual appointment may be provided. A virtual appointment window may be loaded in a browser and it may be determined whether a provider has started the virtual appointment. If the virtual appointment has not been started, an enhanced virtual waiting room interface may be loaded. A request for content may be communicated to a content server. Any matched content may be retrieved from the content server. The matched content may be presented through the enhanced virtual waiting room interface. The enhanced virtual waiting room interface may be transitioned to the virtual appointment when a provider joins or starts the virtual appointment.

### BRIEF DESCRIPTION OF THE FIGURES

Advantages of embodiments of the present invention will be apparent from the following detailed description of the exemplary embodiments thereof, which description should be considered in conjunction with the accompanying drawings in which like numerals indicate like elements, in which:

FIG. 1 shows an exemplary embodiment of an enhanced virtual waiting room;

FIG. 2 shows an exemplary embodiment of an enhanced virtual waiting room;

FIG. 3 shows an exemplary embodiment of an enhanced virtual waiting room;

FIG. 4 shows an exemplary embodiment of an enhanced virtual waiting room;

FIG. 5 shows an exemplary embodiment of an enhanced virtual waiting room;

FIG. 6 shows an exemplary embodiment of an enhanced virtual waiting room;

FIG. 7 shows an exemplary flowchart depicting an exemplary embodiment of a process for presenting an enhanced virtual waiting room.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequence of actions described herein can be considered to be embodied entirely within any form of computer readable storage medium having stored therein a corresponding set of computer instructions that upon execution would cause an associated processor to perform the functionality described herein. Thus, the various aspects of the invention may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

According to an exemplary embodiment, and referring generally to the figures, an enhanced virtual waiting room 100 may be disclosed. An enhanced virtual waiting room 100 may be presented to a user while waiting for a video and/or audio teleconference to begin. The enhanced virtual waiting room may present its audience with curated media and advertising. The media and advertising may be specialty specific according to specialties of the individual appointment provider or the provider's practice or company. According to some exemplary embodiments, advertising content request may specifically exclude reasons for the appointment, and/or user/patient data and history or protected health information.

Once a user, invitee, or patient engages an appointment link, they may be directed to the virtual waiting room, which may initiate the video conference once the parties have joined. According to some exemplary embodiments, the video session facilitated through the virtual waiting room may be initiated immediately, upon at least two parties joining, once all parties have joined, or upon initiation by a provider. A virtual waiting room may be entered when accessing a scheduled appointment and awaiting other participants or when a user schedules to be seen immediately. According to some embodiments, a virtual waiting room may be presented until a provider selects to start an appointment and/or when a provider or user leaves an appointment or temporarily puts an appointment on hold. Furthermore, consents, documents for electronic signature or other informational documents may be accessed and/or completed by a provider, patient, or user in the virtual waiting room.

The enhanced virtual waiting room 100 may improve user/patient retention and appointment success. If a patient is not engaged while awaiting a virtual appointment, the patient or user may leave the waiting screen prior to the appointment, which could result in a missed appointment for both the patient/user and the service provider.

According to some exemplary embodiments, advertisements may be presented through a banner displayed on a user screen. The banner may include video and/or static advertisement data. In addition to or instead of banner advertisements, some exemplary embodiments of an enhanced virtual waiting room 100 may include a user interface presenting a library of curated content The curated content may also include advertising and promotional content or content which is primarily intended for entertainment purposes. The curated content may be in the nature of documents and forms, videos, images, audio records, articles, publications, television shows, movies, games, websites, or online applications. Traditional office waiting room content including health and wellness videos, educational videos, TED talks and advertisements may be provided. In embodiments employing a content library, advertisements may still be presented through adjacent banners or may be presented in a content screen during or prior to content playback, as would be understood by a person having ordinary skill in the art. Content may refer to the entire experience presented through the enhanced virtual waiting room window.

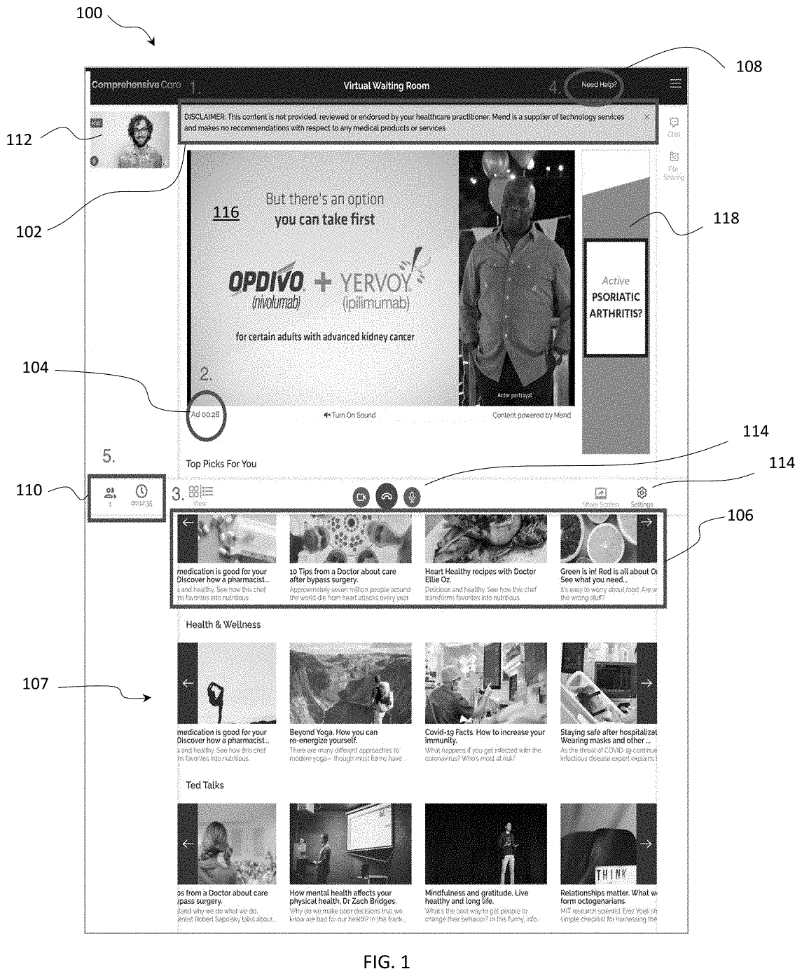

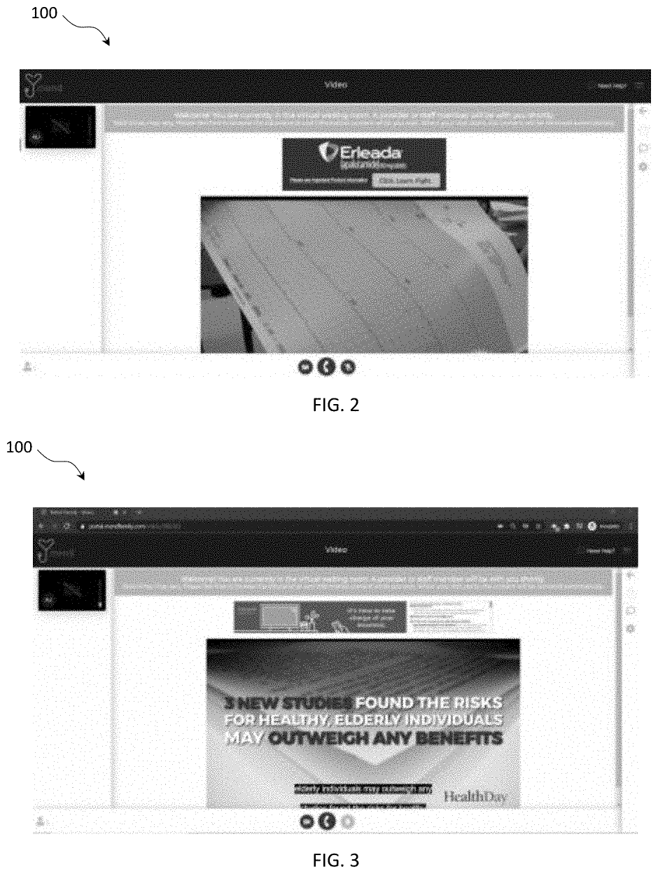

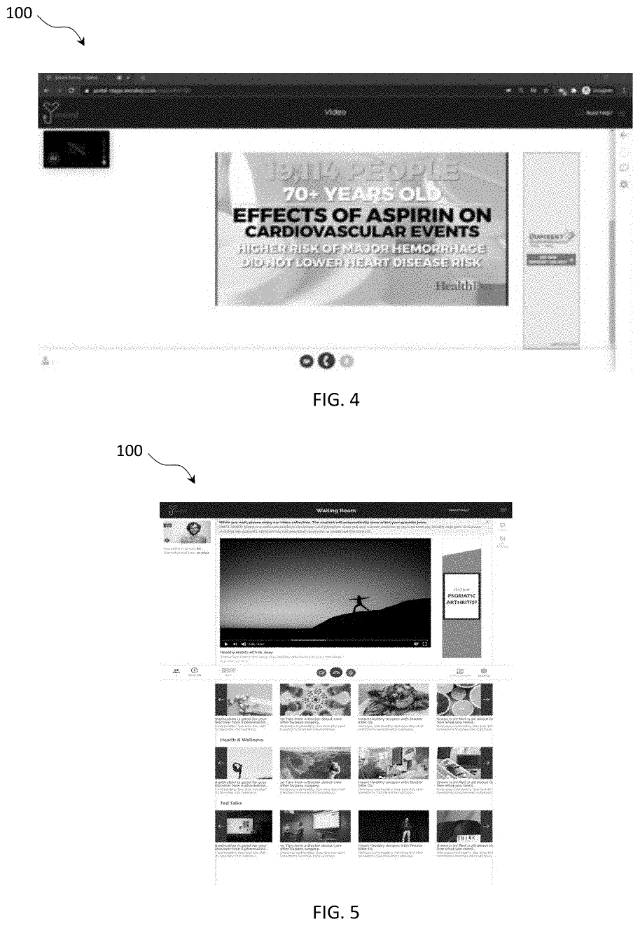

Now referring to exemplary FIG. 1-4, an enhanced virtual waiting room 100 may be provided. Waiting room 100 may include a disclaimer box 102, which may inform users that content is not a direct recommendation or referral from a provider and that the enhanced virtual waiting room developer/operating entity makes no product or service recommendations. Waiting room 100 may also include an advertisement countdown 104, which may identify content as an advertisement and which may provide a countdown clock indicating when an advertisement will conclude or when a user may select to bypass or skip the remaining portion of an advertisement. Waiting room 100 may include a selection of provider content 106 or videos supplied by a provider organization. Provider content 106 may optionally be prioritized over all other non-ad content on a content library interface, for example, by providing organization specific content on a first line of content. Curated content and/or general content 107 provided from the content server may be provided below provider content 106. A live support feature 108 may also be provided through an enhanced virtual waiting room 100. The live support feature 108 may allow a user to contact and obtain customer support or technical support. Waiting room 100 may also provide a wait time counter 110, which may initiate a timer feature when the virtual waiting room is loaded and may show the length of time in the virtual waiting room 100 in real-time. According to some embodiments, a small window or picture-in-picture 112 within the virtual waiting room may show the view through the respective user's camera displayed to the other meeting participant(s). Controls for in-meeting actions 114 and preferences may optionally be available while waiting. Waiting room 100 may further include a content viewing window 116 and may optionally include at least one ad banner 118. According to some exemplary embodiments, an estimated wait time may also be calculated and presented through the enhanced virtual waiting room interface.

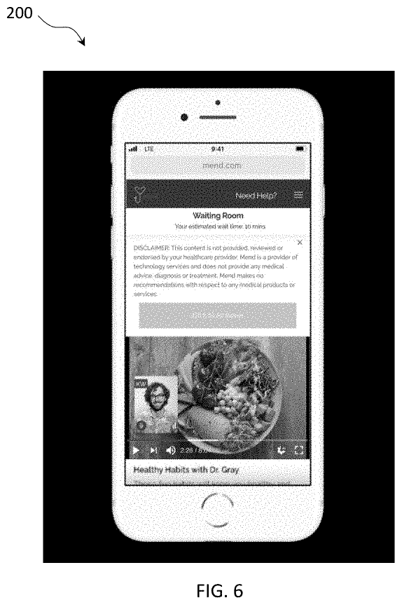

Exemplary FIG. 5 may show an embodiment of an enhanced virtual waiting room 100 presented on a desktop browser or software application. Exemplary FIG. 6 may show an embodiment of an enhanced virtual waiting room 200 presented on a mobile browser or software application.

An enhanced virtual waiting room 100 may be automatically closed when a provider joins a scheduled virtual appointment and the user may automatically be redirected to display a virtual appointment interface. The virtual appointment interface may be provided on the same website or application page or portion thereof as the virtual waiting room. According to some exemplary embodiments, content viewed by a user may be paused during a virtual appointment and content status data may be saved by the enhanced virtual waiting room system and may be utilized to resume the content when a provider leaves a virtual appointment or pauses a virtual appointment. Therefore, if a provider needs to temporarily leave a virtual appointment, the user or patient may continue engaging content in the virtual waiting room 100 during the temporary break in the appointment. According to some further exemplary embodiments, a user may be able to access content after the conclusion of a virtual appointment. For example, the virtual appointment window may transition back to the enhanced virtual waiting room.

According to some embodiments, targeted content may be curated based only on publicly available information including provider specialty, location, and National Provider Identifier (NPI) records. There may be one or more content and advertisement server, which may be isolated from patient data servers or databases. The patient data may include protected health information (PHI) and by isolating the patient data, communication and/or disclosure of PHI may be prevented. When content or advertisements are uploaded to a content server, they may be categorized with metadata or tags, such as a practice specialty or location. The tag may then be used to target content to users in a virtual waiting room based on matching content tags to appointment provider data including specialty, location, and NPI records. For example, a virtual waiting room system may call content from a content server using only provider specialty and location in the request. Provider data may be kept in a provider database. The content server may return a selection of content, which may include advertisements, based on the request. The returned selection of content may include general content and/or matched content based on a matched specialty, location, or provider NPI records. Still further, the returned content may include content created or provided by the individual provider for the virtual appointment or the provider's practice/company. This content may be matched based on provider data and content metadata. According to some embodiments, all content created or provided by a specific provider may always be presented from a content server when content is called by a virtual waiting room for that provider. The enhanced virtual waiting room 100 may also support content and/or subtitles in multiple languages. Still further, banner or video advertisements may also include important safety information notices.

An enhanced virtual waiting room platform may optionally record impressions or views of content, organization ID associated with an impression, provider specialty, and provider NPI data. According to an exemplary embodiment, no patient data, appointment data, or protected health information may be used for retrieving content or may be provided to content sponsors or providers.

According to some further embodiments, the enhanced virtual waiting room 100 may provide tech support capability. Technical difficulties, such as a poor connection, may be determined when a user accesses an enhanced virtual waiting room 100 and steps may be taken to resolve any technical issues. A technical help desk may be provided through an enhanced virtual waiting room 100, which may allow service providers to focus on delivering appropriate services or care during the scheduled appointment rather than providing tech support. This may not only improve efficiency for users and providers of virtual appointments, but also improve the efficiency and connectivity of the virtual communication platform, reducing lag and other disruptions.

To assist users with tech support while connected to an enhanced virtual waiting room, chat bots, which may be operated using Artificial Intelligence (AI), may offer solutions if connectivity issues or other technical problems are identified by the system or a user. The tech support function and/or bot may automatically check user connections and may proactively recommend common fixes through the virtual waiting room 100 and/or chat with a user to resolve common technical issues. Connections may include internet connection and audio or video connections. Fixes may include troubleshooting a WiFi connection, audio device connection, or video device connection. The tech support function may cause a user's computer to present notifications to a user, download and/or run troubleshooting software, seek permissions to download or run troubleshooting software, and seek permission to or run various computing commands. The commands may include turning on a connection, resetting a connection, adjusting volume and/or mute controls, instructing a user to check physical connections, searching, notifying a user to disable, and/or disabling firewalls, antivirus software, VPN clients, and malware that may impact a connection. Some or all of the tech support functions may be performed by a tech support bot or by a live tech support agent, who may be connected through the enhanced virtual waiting room. The tech support through the enhanced virtual waiting room may enable a live screenshare and/or may grant remote access and control of a user device to a tech support agent.

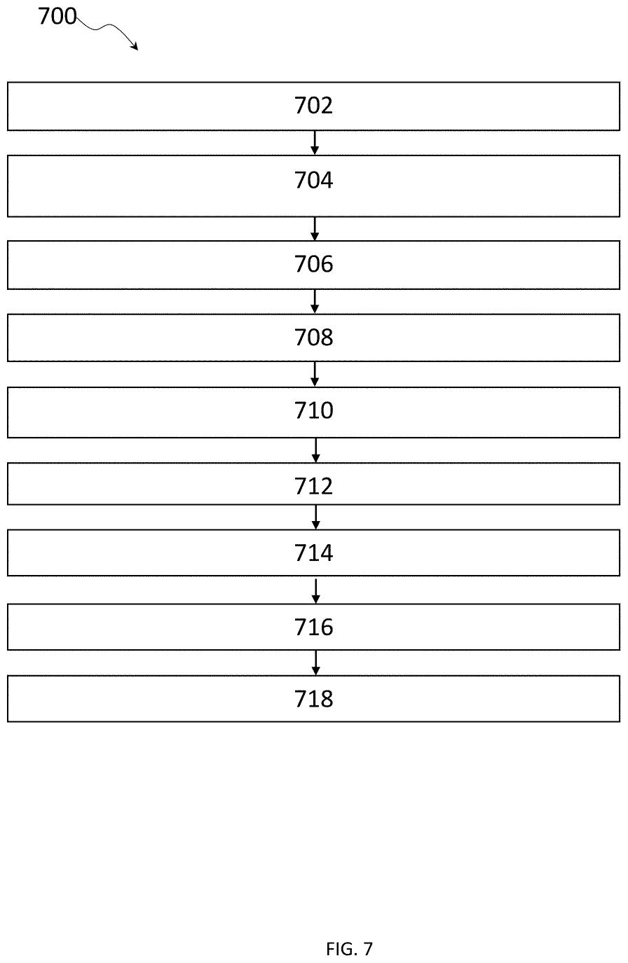

Now referring to exemplary FIG. 7, a method for providing an enhanced virtual waiting room 700 may be provided. According to an exemplary embodiment, a user may click or engage a virtual appointment link 702, which may launch a virtual appointment page in a browser or application 704. The virtual appointment page may present an interface for performing an audio or video conference. The system may check to see if a provider has started the virtual appointment 706. If the provider has started the virtual appointment, the virtual appointment may be initiated on the page 708. If the provider has not started the virtual appointment, the user may be presented with an enhanced virtual waiting room 710. The enhanced virtual waiting room system may automatically check a user's connections, including internet, audio, and video 712. The system may utilize bots to check and troubleshoot technical issues. If a technical issue cannot be resolved, the system may utilize a live help feature to connect the user to a live tech support agent, as discussed above. When no technical issues are identified or when no technical issues are actively being resolved, the enhanced virtual waiting room may present a user with curated content 714. A virtual appointment system may have access to one or more databases with provider information and user/patient information and may utilize this information to present a user with documents or forms supplied by a provider. The virtual appointment system may also utilize an isolated content server, from which the enhanced virtual waiting room may call content or advertisements 716 using data, such as provider specialty, location, and other provider information, as discussed above. The system may not call content or ads using protected user or patient information. According to some embodiments, a content server may be separate from an ad server. When a provider initiates the virtual appointment, the enhanced virtual waiting room content may automatically be paused, closed, or hidden and the virtual appointment may be started 718.

According to some alternatively exemplary embodiments, as would be understood by a person having ordinary skill in the art, the content requests may or may not include information about the provider conducting the visit or the user/patient. A provider's NPI, specialty, location, or other targeting details may be used for matching content. Patient or user demographic data, gender, type of appointment, reason for appointment, age, and other patient or user specific data may also optionally be used to match content. Still further, the patient/user data may optionally be de-identifiable data.

According to additional exemplary embodiments, content may be presented at various points in a patient or user experience, including in-person and virtual touch points. These touch points may include check-ins, confirmations, reschedules, cancellations, new appointment scheduling, digital forms, checkout or other touch points, as would be understood by a person of ordinary skill in the art. These touch points may be engaged through an in-person kiosk, a web browser, a mobile application, monitors or televisions in an in-person environment, or over an audio or audiovisual communication line, such as a telephone line. The touch points may triggered at points of a user journey, including a search phase, a provider listing or directory, provider search, transparency and reputation management, scheduling or on demand appointments, low acuity navigation, triage, virtual visits, in-person visits, digital wayfinding, referral management, patient engagement, care coordination and navigation, easy bill payment, and community connection. The content may optionally be presented through third party software, webpages and devices along the user journey.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art (for example, features associated with certain configurations of the invention may instead be associated with any other configurations of the invention, as desired).

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for engaging users waiting for a virtual appointment comprising:
accessing a virtual appointment window in a browser;
determining whether a provider has started the virtual appointment;
loading an enhanced virtual waiting room interface in the virtual appointment window when the virtual appointment has not been started;
communicating a request for content to a content server;
retrieving matched content from the content server;
presenting the matched content through the enhanced virtual waiting room interface; and
transitioning the enhanced virtual waiting room interface to the virtual appointment when a provider joins or starts the virtual appointment.

2. The method of claim 1, further comprising automatically checking for technical issues on the user device, presenting a notification on the user device, and troubleshooting the technical issues, if found.

3. The method of claim 1, wherein the request for content comprises at least one of provider data or user data.

4. The method of claim 3, wherein the request for content comprises at least one of provider specialty, provider location, and National Provider Identifier records.

5. The method of claim 1, wherein the content server is isolated from user data.

6. The method of claim 1, further comprising detecting a user internet connection status.

7. The method of claim 1, further comprising detecting at least one of a user audio device or user video device connection status.

8. The method of claim 1, further comprising connecting a user to a live technical support agent, when a chatbot is unable to resolve a technical difficulty.

9. The method of claim 1, further comprising presenting at least one advertisement banner or video ad in the enhanced virtual waiting room interface.

10. The method of claim 1, further comprising calculating an estimated wait time and presenting the estimated wait time through the enhanced virtual waiting room interface.

11. The method of claim 1, wherein the content is an advertisement.

12. The method of claim 1, wherein the content is created or supplied by the provider for the virtual appointment.

13. The method of claim 1, further comprising transitioning the virtual appointment window to the enhanced virtual waiting room content when a provider leaves or ends the virtual appointment.

14. The method of claim 1, wherein the content is educational or entertainment content.

15. The method of claim 1, further comprising presenting content at patient touch points during an in-person or virtual patient visit.
